(19) United States
(12) Patent Application Publication
(10) Pub. No.: US 2008/0176317 A1
(43) Pub. Date: Jul. 24, 2008

(54) System and device for transformation and detection of substances

(76) Inventors: Kirollos S. Kirollos, Virginia Beach, VA (US); Gueorgui Milev Mihaylov, Virginia Beach, VA (US); Bryan I. Truex, Belleair Beach, FL (US)

Correspondence Address: Bradley D. Goldizen, One Columbus Center, Ste. 665, Virginia Beach, VA, 23462, US

(21) Appl. No.: 11/787,327

(22) Filed: Apr. 16, 2007

Related U.S. Application Data
(60) Provisional application No. 60/792,373, filed on Apr. 17, 2006.

(51) Int. Cl. C12M 1/34; B01J 19/00; G01N 21/01; G01N 21/78; G01N 23/00; G01N 21/64; G01N 27/00; G01N 27/26

(52) U.S. Cl. 435/288.7; 422/68.1; 435/287.1; 422/82.08; 422/82.11; 422/55; 422/82.02; 422/82.12; 204/400; 422/82.05; 250/306

(57) ABSTRACT

A system and method for transformation of at least one harmful analyte that is difficult to detect to a substance or substances that are more readily detectable by a detector unit and includes parts and steps for detection and quantification of a target analyte or a plurality of analytes. The system includes a transformer having a reagent that transforms the at least one harmful analyte to a specific substance that is capable of being detected by an available sensing device. The system includes an end of service life indicator. Information relating to the transformer may be included on a housing.

This application claims priority from U.S. Provisional Patent Application Ser. No. 60/792,373, filed on Apr. 17, 2006.

The subject matter of the present invention did not receive federal government research and development funding.

### BACKGROUND OF THE INVENTION

The present invention generally relates to a system and method for detection of substances. More particularly, the present invention relates to a system and method for the detection and quantification of target analyte and includes a transformer capable of transforming a specific substance to another substance. The transformed substance is capable of being detected by an available or currently existing sensing device. Moreover, the system may trap undesired substances while allowing other substances to pass through and to be detected by an available sensing device.

The need for quick and real time detection of toxic substances is of great importance to the health of humans and the environment. The current state of the art and the market have a wealth of portable and hand-held devices capable of quick and real time detection of substances that are available. These devices depend on sensors that operate on different principles which can be generally categorized as electrochemical sensors, mass sensors and optical sensors and are available types of sensors.

Electrochemical sensors operate by measuring a potential difference or voltage (potentiometric), quantifying an electric current (amperometric) or quantifying conductivity (conductometric). All these sensors rely on absorption of the target analyte onto a sensor and a change of physical and/or chemical properties in a reversible fashion. This leads to two main drawbacks. First, there are a limited number of groups of substances that are detectable. Second, the sensors are incapable of specifically distinguishing between different substances within a group that has similar chemical and physical properties. Thus, existing sensors are sometimes unreliable and may lead to false reporting of detected substances.

To overcome the aforementioned limitations, other devices must be used to achieve a wide diversity and a higher specificity of substances detection and quantification. Typically, these other devices are based on technologies such as gas chromatography, liquid chromatography and spectroscopy. However, these devices are expensive, bulky and require well trained personnel to operate them. These limitations hinder the use of chromatography and spectroscopy devices in the field and render their use impractical. The concept of having a substance selective detection system is the subject of several patents. Colorimetric devices were the subject of such systems. Grosskopf (U.S. Pat. No. 3,99,973) teaches a hopkalite-like pre-filter to eliminate carbon monoxide from mixture of gases that can adversely affect the reaction in the calorimetric tube. Leichnilz (U.S. Pat. Nos. 4,230,457 and 4,259,287) teaches a means to convert aerosols into measurable gaseous substances and measure the aerosol content by measuring the proportion of the generated gases. Kretshmer (U.S. Pat. No. 4,022,578) uses a similar approach, where the first layer in the detector tube is impregnated with reagent capable of oxidizing halogenated hydrocarbons to measurable halogen in the second part of the tube. Kramer et al. (U.S. Pat. No. 4,789,638) describes a multi-zone device for hydrazine and hydrazine derivatives, which uses one part of the tube-like device as a reactor zone transforming the analyte to halogen and then detecting the halogen in another zone. In all, these patents that relate to colorimetric tube-like devices, the converting, trapping or transforming part has a length adjusted by trials and errors to accommodate a single use, such that the detector tubes are one-time use devices. Therefore, there is no requirement of controlling or observing their converting, trapping or transforming power. This approach has been in use in calorimetric gas measuring device for fluorine in May (U.S. Pat. No. 4,933,144). In the May patent, the converting media is used to generate a secondary substance identifiable by a detector layer, in a tube-like or moving layer format. In the second case, there is no means to control and observe the dissipation of the converting power advancing with the time and quantity of the converted substance, i.e. fluorine. In U.S. Pat. No. 4,197,177, two stages of analyte preparation are applied, the first is humidity removal and the second is conversion of nitrogen oxides to nitrogen dioxide and consecutive detection of the nitrogen dioxide by an electrochemical sensor. None of the filtration and conversion layers has means to observe the anticipated depletion. In order to make more selective inherited non-specific sensors such as a photo-ionization detector, U.S. Pat. No. 5,654,498 to Kessel or electrochemical sensor, U.S. Pat. No. 6,840,084 to Nikolskaya, include converting layers that are adjusted to deliver to the sensor air flow with the desirable component in a continuous time regime. In both cases, the converting layer is affected and depleted by the humidity and the satellite gases or vapors to be converted and there is no means to observe the depletion of the converting material.

### SUMMARY OF THE INVENTION

The present invention is a system and method for the detecting and quantifying target analytes. The system includes a transformer capable of transforming a first substance to a second substance. The first substance is difficult, if not impossible, to detect by existing or available detectors; whilst the second substance is readily detectable by an available sensing device such as the previously mentioned, existing electrochemical sensor. The transformer is capable of localizing and trapping undesired satellites of target analyte from passing through and being detected by an available sensing device. The available sensing device detects and quantifies the transformed analyte that passes through the transformer. An end of service life indicator includes a selection of one or more substances to perform chemical change and/or biochemical change and/or physical change to indicate the depletion and/or progress and/or end of service life of the transformer. A pump draws fluids through the transformer and sensor to aid in the detection process.

It is an objective of the present invention to provide a new and improved device for protecting humans in the workplace from harmful substances by readily detecting and quantifying these substances.

It is another objective of the invention to provide a means of selectively detecting and quantifying a wide range of toxic substances in fluids.

Another objective of the invention is to provide a simple low cost device and method for selectively detecting and quantifying a wide range of toxic substances in fluids.

Yet another objective of the present invention is to provide a handheld device to detect and quantify a wide range of toxic substances in the field.

In accordance with one aspect of the invention, the foregoing and other objectives are achieved by providing a transformer capable of transforming a specific substance to another substance capable of being detected by a sensing device readily available in the marketplace. Moreover, the invention bolsters the capabilities of existing sensors to accurately detect harmful substances.

Another aspect of the invention is achieved by providing a transformer capable of localizing and trapping undesired satellites of target analyte from passing through and being detected by currently available sensors that are readily purchasable.

Another aspect of the invention is achieved by providing a collecting means to move fluids through the transformer. The collecting means may be a pump that collects more analyte and increases the sensitivity of the detection. A means to measure ambient temperature and humidity may also be provided to optimize the performance of the detection system. Further, a means that heats the transformer to a preset temperature may be provided.

In another embodiment, a suction pump having a log system which includes a programmable microprocessor control system comprising a memory having a program for executing instructions and containing relevant information. Personal computer-based built-in software provides programming and relevant sampling information unique to a specific transformer application. This information may include pumping flow rate, battery charge, time, temperature, humidity, as well as correction factors for aiding in detecting and quantifying the target analyte and atmospheric pressure, barometer or the like. The correction factors may be provided in way of a database stored in an internal memory.

The transformer can incorporate an EE PROM, E square or read/writable or other Radio Frequency Identification (RFID) tag or other programmable memory device that stores the following information: part number, serial number, manufacturer's lot number, application name, range, date and time of each alarm, start date and time of each service date, date and time for each end of service time, type of product, the cumulative exposure to the analyte, usage times, dates and durations, manufacturing date, shipping date, unit of measure, flow rate, flow duration, password or interface code and expiration date. The memory chip may be removable and the memory cleared such that the device may be reused.

Additionally, the following information can be stored on or in the transformer as a barcode; part number, application name, product type, serial number, manufacturer's lot number, range, type of product, unit of measure, flow rate, flow duration and expiration date. The transformer can download the data to the sensor or the RFID reader or a computer and/or receive uploaded information from the sensor or a computer or from the RFID reader.

The transformer housing can be made from metal, glass, clear or opaque plastic such as polypropylene, polyethylene, polyesters, and/or Teflon.

The transformer has a single or a plurality of layers incorporating chemical and/or biochemical formulations capable of transforming the target analyte to another substance that is detectable by a readily available sensor.

The transformer comprises at least one layer incorporating chemical and/or biochemical formulations capable of localizing and/or trapping undesired satellites of the target analyte from passing through to be detected by the available sensors mentioned above. The chemical and/or biochemical formulations may include formulations that undergo colorimetric, fluorescent, luminescent or bioluminescent responses upon reacting or interacting with the target analyte.

The transformer may further include a layer that functions as an end-of-service-life-indicator (ESLI) to provide early indication of the consumption and/or saturation.

In one embodiment, the ESLI layer is located next to a layer having a reagent that reacts with the target analyte to convert it to a more readily detectable substance. That is, the single or multiple layers that incorporate chemical and/or biochemical formulations are capable of transforming the target analyte to another substance that is detectable by an available sensor.

The ESLI layer may be arranged next to a buffer layer. This buffer layer acts as safety buffer and can be constructed from the same materials as the single or multiple layers incorporating chemical and/or biochemical formulations capable of transforming the target analyte to another substance that is readily detectable by an available sensor. Additionally, the buffer layer can be constructed from the same materials as the single or plurality of layers incorporating chemical and/or biochemical formulations capable of localizing and/or trapping undesired satellites of the target analyte from passing through to be detected by the available sensors.

The ESLI layer undergoes characteristic changes upon exposure to the transformable target analyte or to the undesired satellites of the target analyte. These changes in the characteristics may include optical, electrical, pyroelectric, piezoelectric, any combination thereof and/or any measurable change in a characteristic of the ESLI layer.

The optical, electrical, pyroelectric, piezoelectric and/or any measurable change in a characteristic can be measured visually and/or with any available detectors and sensors capable of detecting and/or measuring a chemical and/or the physical change of the properties and characteristics of the ESLI.

An improvement of the transformer of the present invention may be included, for example, a construction of the end of service life indicator and the transformer as an integral unit. In this example, the analyte moves through single or multiple layers to react with the chemical and/or biochemical formulations. The extent of the analyte's movement through the reagent material can be ascertained and the consumption of the reagent material is determined by a color change, fluorescence and/or illuminations of the reacted reagent material as the fluid analyte moves through the layer(s). One or more of the layers are inert to the analyte as the fluids moves through, therefore it does not change color, and/or does not fluoresce or illuminate when exposed to light, ultraviolet light, infrared light and the like. The material in the inert layer has a contrasting color to the reactive materials. Otherwise, the inert material can have a non-contrasting color to the reactive material. This may be the case with fluorescent, luminescent or bioluminescent reagents. As the fluid analyte passes through the inert layer, there is no reaction, no color change, no fluorescence, and no illumination. The inert layer forms a clear line of demarcation between the consumed material and the unconsumed material. By reading the amount of reacted material, it is possible to estimate the remaining useful life of the transformer. Once the analyte passes through the inert layer, the analyte comes in contact with the reactive layer or multiple active layers and changes color or fluoresces or illuminates, as described above. There can be one or more inert layers in the transformer to measure the progress of the analyte moving through the entire transformer or a just a portion of the transformer.

The transformer allows fluids to pass through with optimized resistance to achieve high capacity, as well as uniform and fluent flow of fluids.

In the preferred embodiment, the transformer best functions at temperatures ranging between 0° F. to 105° F. Operation at lower temperatures can be achieved by incorporating a heater. Operation at higher temperatures is usually accompanied by a higher reaction rate, in this case correction factors can be applied. The correction factor data can be supplied in tables or it can be incorporated in an EE PROM, E square or read/writable or other Radio Frequency Identification (RFID) tag or other memory device or bar code on the transformer and/or the pump and/or the sensing device.

The chemical and/or biochemical formulations include a support substrate that has a macro porous structure to support and encapsulate the chemical and/or biochemical active ingredients. The macro porous member includes micro porous and/or nonporous member to support and encapsulate the chemical and/or biochemical active ingredients.

The chemical and/or biochemical formulations may include a binding substrate to bind and/or localize the chemical and/or biochemical active ingredients to the micro porous and/or nonporous support member. The micro porous and/or nonporous support member can be silica, alumina, zeolite, porcelain, molecular sieves or any similar inert materials that are compatible with the chemical and/or biochemical active ingredients.

The transformer of the present invention includes variations of chemicals and/or biochemical formulations capable of localizing and/or trapping individually the following substances while allowing other substances to pass through intact. The trapped substances may include Ammonia, Aromatic hydrocarbons (such as toluene, xylene), Br₂, Carbon tetrachloride, Cl₂ CO₂, H₂S, HCl, HF, Lower class hydrocarbons (C₁ through C₆), Mercaptans, Nitric Acid, Nitrogen oxides NO₂, Organic gases and vapors, O₃, SO₂, SO₃, Unsaturated halogenated hydrocarbons and water vapor.

An example of a possible formulation capable of localizing and/or trapping individually the following substances while allowing other substances to pass through intact is a chemical transformer that includes alkaline glass fibers which react with HF while allowing other substances including other acids to pass through intact. This formulation is especially useful in localizing and/or trapping HF. For H₂S, the transformer may include lead acetate which reacts with H₂S and allows other substances pass through intact.

Yet another example of a possible formulation capable of localizing and/or trapping individually the following substances while allowing other substances to pass through intact is a chemical transformer that includes chromium oxide and sulfuric acid. The oxidation potential of these two chemicals can be adjusted to react, and hence, trap toluene, xylene and other hydrocarbons while allowing benzene to pass intact and be detected selectively by a commercially available sensor. The transformer includes an ESLI to indicate the progress in consumption of the active trapping layer, a buffer layer to ensure that none of the undesired substances passes through to the sensor and a pre-layer of dryer to remove moisture and hence increase the capacity and efficiency of the transformer.

The transformer of the present invention includes variations of chemical and/or biochemical formulations capable of transforming individually the following target analytes to other substances and/or generating, due to the presence of the individual following target analytes, other substances that are readily detectable by available sensors while allowing other substances to pass through intact. Some of these target analytes include Acetaldehyde, Acetone, Acetylene, Acrolein, Acrylonitrile, Arsine, Benzene, Tert-Butyl Mercaptan, Carbon Disulfide, Carbon Monoxide, Carbon tetrachloride, Carbonyl Sulfide, Chlorobenzene, Chloroform, Diborane, o-Dichlorobenzene, 1,2-Dichloroethylene, Ethylene Glycol, Ethylene Oxide, Ethyl Mercaptan, Formaldehyde, Hydrocarbons (Aliphatic), Hydrogen Cyanide, Mercaptans, Methacrylonitrile, Methyl Bromide, Nitrogen Oxides, Phosphine, Stoddard Solvent, Sulfur Dioxide, Tetrachloroethylene, Toluene, 1,1,1-Trichloroethane, Trichloroethylene, Vinyl Chloride, Vinylidene Chloride and Xylene.

An example of an appropriate formulation for detection of acetaldehyde includes a chemical transformer of the present invention that comprises (NH₂OH)₃.HCl. Acetaldehyde reacts with (NH₂OH)₃.HCl to generate HCl. HCl is detectable by an HCl portable handheld sensor such as an electrochemical sensor currently available in the marketplace.

An example of an appropriate formulation for detection of acrylonitrile includes a chemical transformer of the present invention that comprises a Cr⁺⁶ compound, H₂SO₄ and HgCl₂. Acrylonitrile reacts with Cr⁺⁶ compound and H₂SO₄ to generates HCN which in turn reacts with HgCl₂ to produce HCl. HCl is detectable by an HCl portable handheld sensor such as electrochemical sensor that is currently available.

An example of an appropriate formulation for detection of chloroform includes a chemical transformer of the present invention that comprises I₂O₇ and H₂S₂O₇. Chloroform reacts with I₂O₇ and H₂S₂O₇ to generate Cl₂. Cl₂ is detected by a Cl₂ portable handheld sensor currently available in the marketplace.

An example of an appropriate formulation for detection of Carbon Disulfide includes a chemical transformer of the present invention that comprises I₂O₇ and H₂S₂O₇. Carbon Disulfide reacts with I₂O₇ and H₂S₂O₇ to generate SO₂. SO₂ is detected by an SO₂ portable handheld sensor currently available. Alternatively, the chemical formulation in the chemical transformer of the present invention may include I₂O₇, H₂S₂O₇ and BaCl₂. Carbon Disulfide reacts with I₂O₇ and H₂S₂O₇ to generate SO₂. SO₂ reacts BaCl₂ with to generate HCl. HCl is detectable by an HCl portable handheld sensor.

The transformer of the present invention may be connected upstream and operate in accordance with of any of the following sensors or combinations thereof: an acoustic IR sensor, an argon ion electron capture detector, a biological sensor, a chemfet, a calorimetric sensor, a conductive polymer sensor, an enzyme sensor, a fiber optic sensor, a flame ionization detector, a fluorescence detector, an immobilization of recombination bioluminescent bacterium sensor, an immunoassay sensor, an infrared coherent laser source sensor, an infrared sensor, an ion mobility detector, an ionization sensor, a laser sensor, a metal oxide sensor, a paper tape sensor, a piezoelectric sensor, a piezopyretic sensor, a SAW sensor, a solid state semi-conductor sensor, a solid state sensor, a solid state thin film semi-conductor sensor, a TiO₂ sensor, a thermal conductivity sensor, a thin film titanium dioxide sensor, a voltametric electrocatalytic sensor and/or a wave guide sensor.

### BRIEF DESCRIPTION OF THE DRAWINGS

FIG. 1 is a simplified schematic representation of one embodiment of the detection system.

FIG. 2 is a simplified schematic representation of another embodiment of the detection system.

FIG. 3A is a simplified schematic representation of another embodiment of the detection system and including a transformer that comprises a liquid reagent supported on a solid material.

FIG. 3B is a schematic of a transformer and showing how a liquid reagent is supported on a solid.

FIG. 4 is a simplified schematic representation of another embodiment of the detection system including a dryer arranged in the system before the transformer. An end of service life indicator (ESLI) is arranged between the transformer and the detector.

FIG. 5 is a simplified schematic representation of another embodiment of the detection system including an ESLI built into the transformer and a heater.

FIG. 6 is a simplified schematic representation of another embodiment of the detection system that includes a radio frequency identifier and reader.

FIG. 7 is still another schematic representation of another embodiment of the invention that includes a transformer having a bar code. A bar code reader is coupled to a data processor for detecting a target analyte.

FIG. 8 is a view of a first embodiment of the transformer and ESLI integrated as one component.

FIG. 9 is a view of a second embodiment of the transformer and ESLI integrated as one component.

### REFERENCE NUMBERS

100. Detection system
106. Dryer
110. Transformer
111. liquid reagent
112. Solid support for the reagent
114. Partially wetted solid support
116. Wetted solid support
120. Detector
130. Pump
140. End of service life indicator —ESLI
141. Housing
142. Reagent layer
143. Buffer layer
144. Inert/clear layer or region
145. ESLI reader
146. Indicator layer
150. Radio frequency identification tag
155. RFID reader
160. Bar-code
162. Bar-code reader
170. Conditioning means heating/cooling
190. Data processor
200. Audible/visual warning means

### DETAILED DESCRIPTION OF THE INVENTION

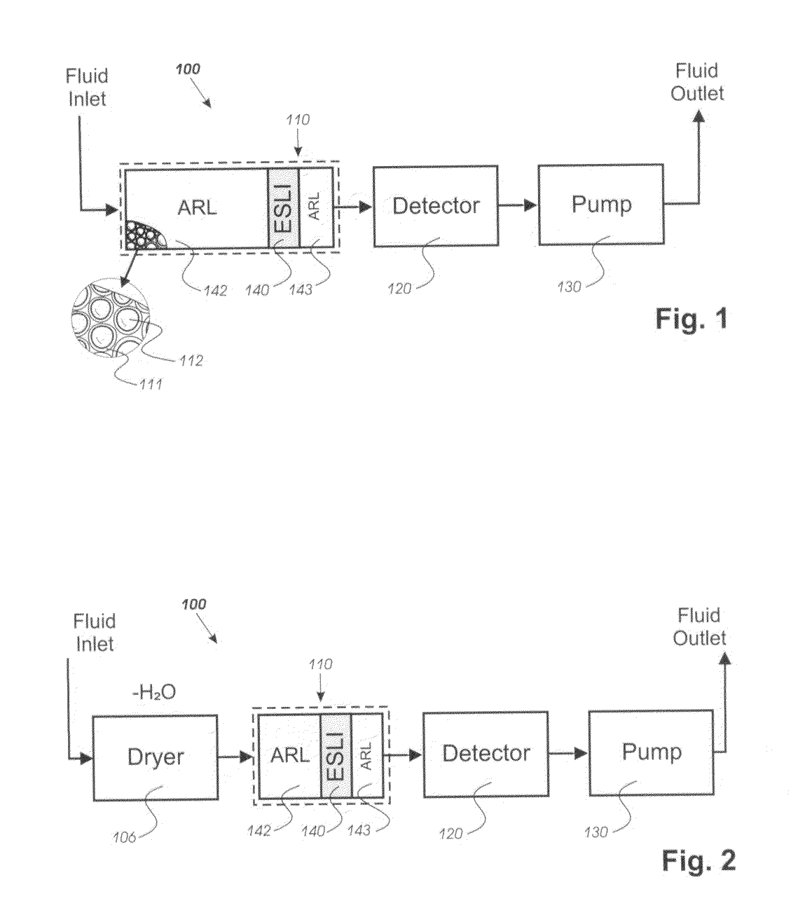

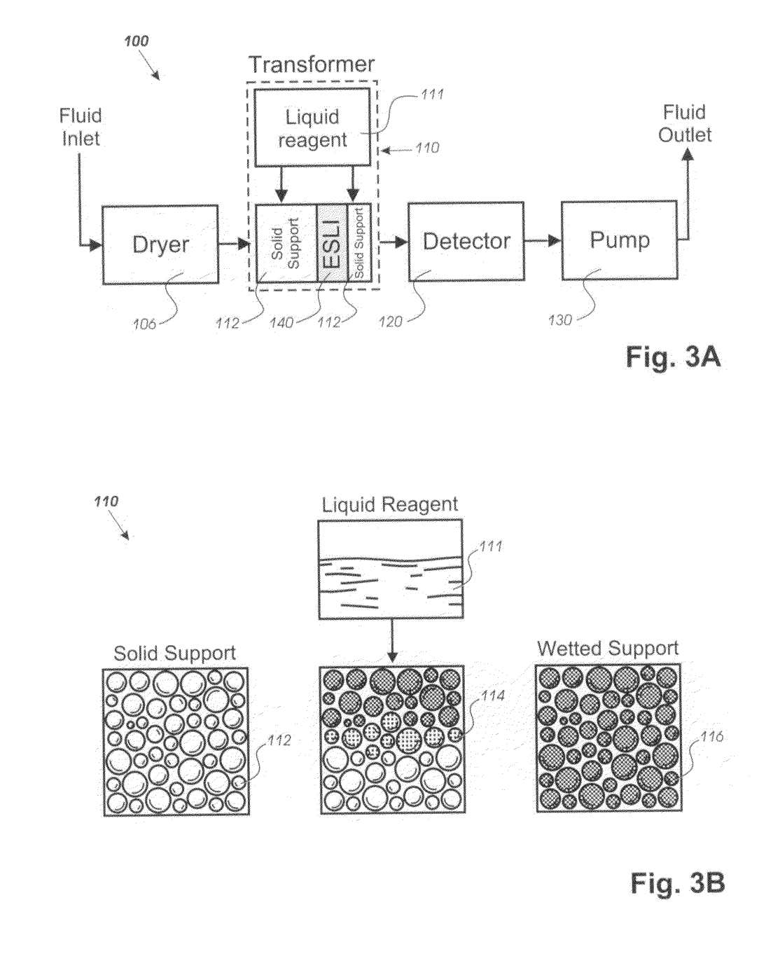

FIG. 1 depicts a first embodiment of the invention. In FIG. 1, a difficult to detect analyte is drawn into the detection system 100 through a fluid inlet and moves through a transformer 110 wherein the analyte reacts with a reagent 111 disposed onto the solid support 112, as shown in FIG. 3B. Both reagent 111 and solid support 112 form reagent layer 142. The reagent may include a binding substrate to bind and/or localize the chemical and/or biochemical active ingredients to the micro porous and/or nonporous support member. The micro porous and/or nonporous support member can be silica, alumina, zeolite porcelain, molecular sieves or any similar inert particulate materials that are compatible with the chemical and/or biochemical active ingredients contained within the transformer 110 to be converted to a readily detectable substance and/or trap undesired substance and allow target analyte to pass through and be detected by available sensor. This detectable substance is easily detected by detector 120, which is preferably a readily available off-the-shelf detection type unit, as mentioned above. A pump 130 is provided for drawing the analyte into the system 100 and through the transformer and detector 120. The end of service life indicator layer 140 separates the transformer into two parts; an analytical reagent layer (ARL) 142 and a buffer (ARL) 143.

The embodiment shown in FIG. 2 is similar to that of FIG. 1 except that a dryer 106 has been added for reducing the moisture content in the target analyte to aid in the transformation of the analyte. The “—H2O” indicia shown above the dryer in respective figures indicates the reduction of the moisture content. The reaction time and amount of reagent necessary to convert the hard to detect analyte to a more readily detectable substance are reduced by lowering the water content in the sampled analyte. Conventional drying substances inert to the analyte can be used in the dryer 106.

The transformer 110 shown in FIG. 3A comprises both a liquid reagent 111 and a solid support 112. The liquid reagent 111 may be disposed and or impregnated onto the solid support 112. The liquid reagent may include a binding substrate to bind and/or localize the chemical and/or biochemical active ingredients to the micro porous and/or nonporous support member. FIG. 3B depicts the mechanism of impregnation of the porous nature of the solid support 112. In the preferred embodiment, a liquid reagent 111 is combined with the solid support 112 to create a wetted support 116. The solid support structure may comprise those types mentioned above, namely micro porous and/or nonporous material. The liquid reagent or the plurality of liquid reagents is added before and or after the collection of a sample. The micro porous and/or nonporous support member can be silica, alumina, zeolite, porcelain, molecular sieves or any similar inert materials that are compatible with the chemical and/or biochemical active ingredients.

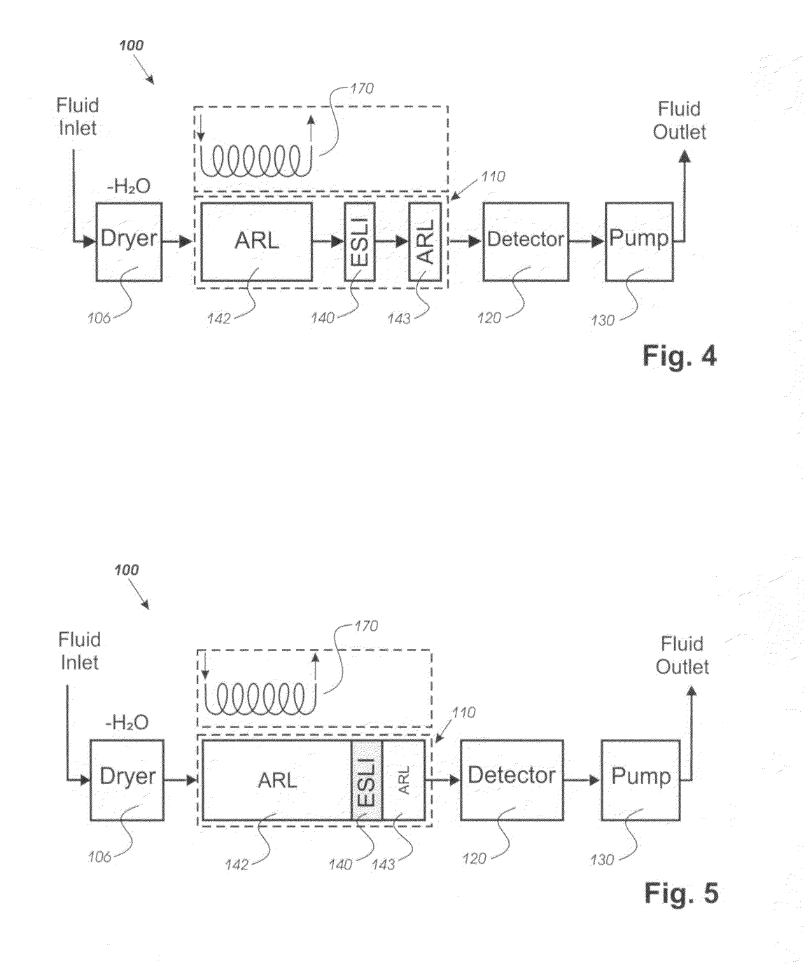

In FIG. 4, an ESLI 140 is a separate unit integrated into the transformer 110. The ESLI 140 is an indicator means that alerts a user when the reagent material of the transformer has been expended. In this manner, the user may determine when the useful life of the reagent material has expired to assure that the system is converting harmful analyte to a more easily detectable substance. Immediately after the ESLI 140, a buffer layer 143 is arranged. The content of buffer layer 143 is the same as the ARL layer 142 or similar to the ARL layer 142.

In FIG. 5, an ESLI 140 is included in the transformer 110 as an integral part thereof. The transformer 110 may include a housing having a clear region (ESLI 140) through which a change in the characteristics of the reagent of the transformer 110 may be easily viewed. The transformer assembly 110 can be heated or cooled by the conditioning means 170 to assure that the transformation process of the hard to detect analyte to a more readily detectable substance occurs at an optimal temperature.

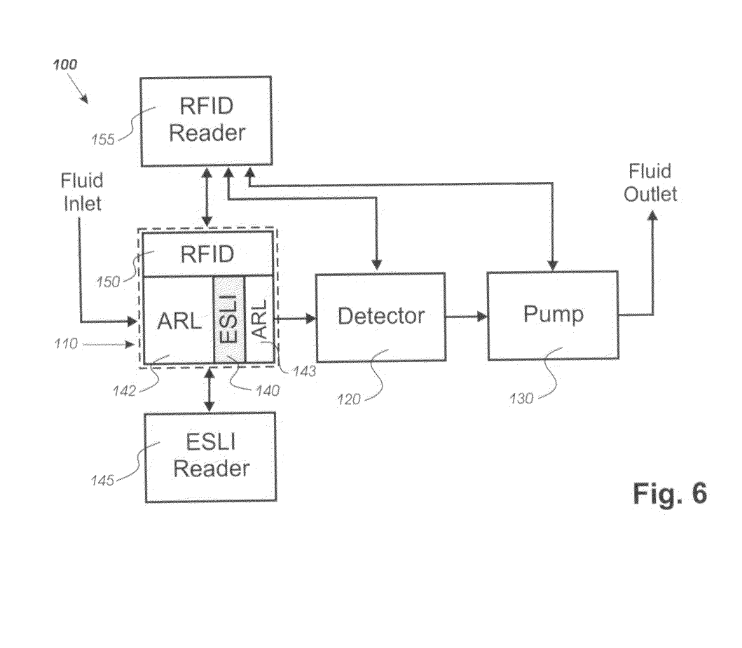

FIG. 6 is an embodiment of the system wherein a transformer is provided with an RFID tag 150 that is attached to the transformer 110. Each RFID tag 150 may include pertinent information relating to the transformer 110 as previously mentioned. An RFID reader 155 reads this pertinent information and inputs it into the detector 120 and pump 130 for providing each with information for controlling the system. It should be noted that other types of sensors may be coupled to the detector for assuring more accurate results. For example, a temperature sensor, a humidity sensor or the like may be coupled to the detector 120, pump 130, ESLI 140 and/or dryer 106.

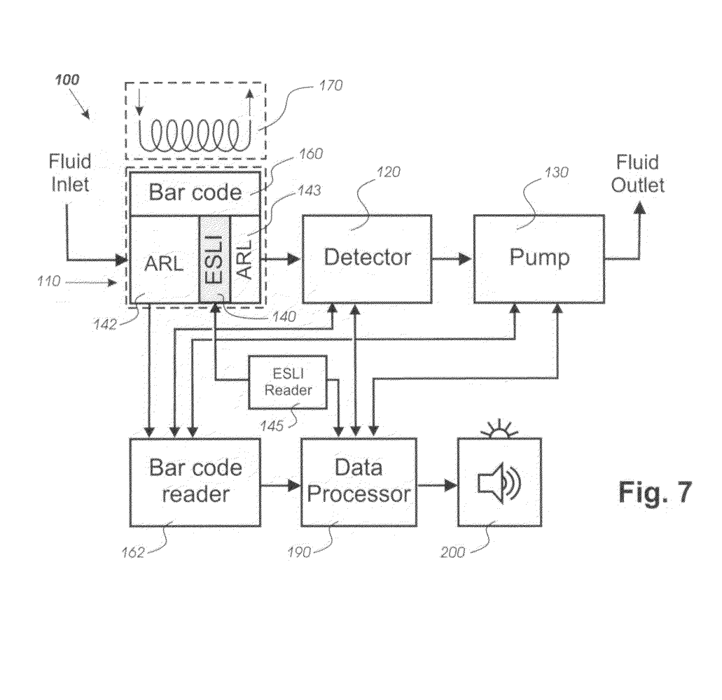

FIG. 7 is another embodiment of the detection system that comprises a bar code 160 that is supplied on the transformer 110. The bar code 160 includes information pertinent to the transformer 110 as discussed previously. A bar code reader 162 decodes the information provided on the bar code 160 and feeds it into a detector 120, a pump 130 and a data processor 190. The information from the bar code reader 162 may be fed only into the data processor 190 which in turn may relay the necessary information to the pump 130 and detector 120. The ESLI reader 145 reads the ESLI 140 and sends information to data processor 190 that generates audible and or visual signal via means 200 to alert a user to any change in the transformer.

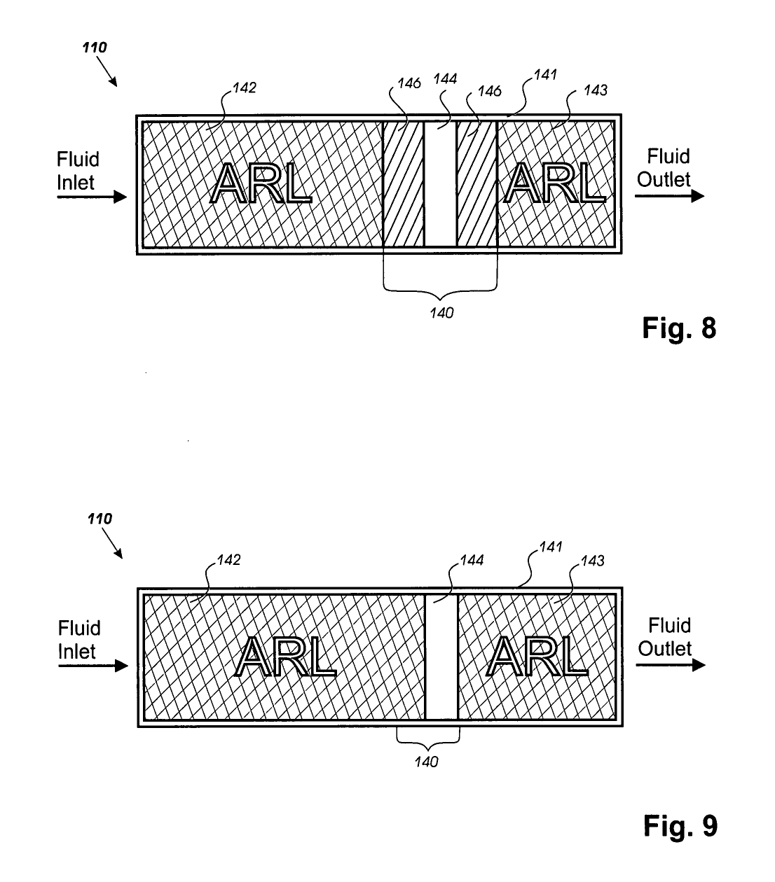

FIG. 8 shows another embodiment of the invention wherein the transformer 110 and ESLI 140 are integrated in one housing 141. In this embodiment, the transformer comprises a reagent layer 142, a buffer layer 143 and an inert/clear region 144. A housing 141 is provided for containing the reagent and buffer material along with the inert material. In this embodiment, the ESLI 140 is represented by the inert/clear layer 144 and the first part of the buffer layer 146 adjacent to the inert layer 144. The transformer housing can be made from metal, glass, clear or opaque plastic such as polypropylene, polyethylene, polyesters, and/or Teflon. The inert layer 144 is a band of material that is inert to the analyte as the fluid moves through it and the material does not change color, and/or does not fluoresce or illuminate when exposed to light, ultraviolet light and/or infrared light. The material in layer 144 is a contrasting color to the reacted material in layer 142 and/or layer 143. The material in layer 144 is not a contrasting color to the material in layer 142, and/or layer 143. This could be the case with fluorescent, luminescent or bioluminescent reagents. As the fluid analyte passes through layer 144, there is no reaction, no color change, no fluorescence, and no illumination. The inert material of layer 144 forms a clear line of demarcation between the consumed material and the unconsumed material. By reading the amount of reacted material, it is possible to estimate the remaining useful life of the transformer. Once the analyte passes through layer 144, the analyte comes in contact with the buffer layer 143 to again react with the reagent material and causing them to change color, fluoresce or illuminate, as described above. There can be one or more “clear areas” (layer 144) in the transformer to measure the progress of the analyte moving through all or a layer of the transformer. The inert/clear layer 144 is sandwiched between two color indicating and or fluorescence and/or illumination layers 146. Layers 146 change color and/or fluorescence and/or illuminate upon contact with the analyte. The layers 146 can be fabricated to interact with the analyte with the same sensitivity or interact with analyte with two different sensitivities. In this embodiment, the ESLI 140 consists of the two layers 146 and the inert layer 144.

FIG. 9 is another embodiment of the transformer 110 and ESLI 140 integrated in one housing 141. In this embodiment, the reagent layer 142 and the buffer layer 143 do not change color, have no fluorescence, and no illumination.

Although the present system, apparatus and device of detecting a target analyte and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, and composition of matter, means, methods and steps described in the specification. For example, various exemplary configurations of a detection system have been described. As one of ordinary skill in the art will readily appreciate from the disclosure of the present invention, various elements from measurement or instrumentation technology, or measuring methods or steps, presently existing or later to be developed that perform substantially the same function or achieve substantially the same result as the corresponding embodiments described herein may be utilized according to the present invention. Accordingly, the appended claims are intended to include within their scope such machines, apparatus, methods, or steps.

We claim:

1. A system for the detection and quantification of target analyte comprising;
a transformer having and fluid inlet and a fluid outlet and capable of transforming a target analyte that is hard to detect to a second substance where the second substance is capable of being detected by an existing sensing device, said first transformer comprising an end of service life indicator to indicate an end of service life of the first transformer;
a sensing device connected to the fluid outlet of the transformer, said sensing device detects and quantifies the second substance to provide an accurate measure of the amount of target analyte taken from a sample; and,
a pump connected to one of the transformer or sensing device to draw the target analyte through the transformer and the second substance through the sensing device.

2. The system of claim 1 wherein said transformer further comprising:
a housing made of one or more substance selected from a group of materials consisting of metal, glass and clear or opaque plastic, said housing surrounding the transformer;
at least one layer arranged within said transformer and incorporating one or more active ingredients selected from a group consisting of chemical and biological substances capable of transforming the target analyte to the second substance detectable by the sensing device and chemical and biological substances capable of localizing and trapping undesired substances and allowing the target analyte to pass through the transformer to be detected by the sensing device;
a solid support substance that has a maco porous member to support and encapsulate the active ingredients and being arranged within said transformer; and,
a memory device selected from an EE PROM, E square, read/writable, Radio Frequency Identification (RFID) tag and barcode being arranged within or on the housing.

3. The system of claim 1 wherein said target analyte includes one or more substances selected from a group consisting of: Acetaldehyde, Acetone, Acetylene, Acrolein, Acrylonitrile, Arsine, Benzene, Bromine, Tert-Butyl Mercaptan, Carbon Disulfide, Carbon Monoxide, Carbon tetrachloride, Carbonyl Sulfide, Chlorine, Chlorobenzene, Chloroform, Diborane, o-Dichlorobenzene, 1,2-Dichloroethylene, Ethylene Glycol, Ethylene Oxide, Ethyl Mercaptan, Fluorine, Formaldehyde, Hydrocarbons (Aliphatic), Hydrogen Chloride, Hydrogen Bromide, Hydrogen Iodide, Hydrogen fluoride, Hydrogen Cyanide, Iodine, Mercaptans, Methacrylonitrile, Methyl Bromide, Nitrogen Oxides, Phosphine, Stoddard Solvent, Sulfur Dioxide, Tetrachloroethylene, Toluene, 1,1,1-Trichloroethane, Trichloroethylene, Vinyl Chloride, Vinylidene Chloride and Xylene.

4. The system of claim 1 wherein said sensing device is selected from a group consisting of Potentiometric sensors, Amperometric sensors, conductometric sensors, Acoustic IR sensors, Argon ion electron capture detectors, Biological sensors, Chemfets, Colorimetric sensors, Conductive polymer sensors, Enzyme sensors, Fiber optic sensors, Flame ionization detectors, Fluorescence detectors, immobilization of recombination bioluminescent bacterium sensors, Immunoassay sensors, Infrared coherent laser source sensors, Infrared sensors, Ion mobility detectors, Ionization sensors, Laser sensors, Metal oxide sensors, Paper tape sensors, Piezoelectric sensors, Pyroelectric sensors, Photo ionization sensors, Saw sensors, Solid state semi-conductor sensors, Solid state sensors, Solid state thin film semi-conductor sensors, TiO₂ sensors, Thermal conductivity sensors, Thin film titanium dioxide sensors, Voltametric electrocatalytic sensors and Wave guide sensors.

5. The system of claim 2 wherein said solid support substance has a macro porous member further comprising one or more selected from a micro porous member and micro nonporous member to support and encapsulate active ingredients that transform the target analyte to the second substance.

6. The system of claim 1 wherein the transformer localizes and traps undesired satellites of target analyte from passing through and being detected by a sensing device.

7. The system of claim 2 wherein said macro porous structure is selected from one or more substances consisting of silica, alumina, zeolite, porcelain, glass, and molecular sieves.

8. The system of claim 2 where in said memory device stores one or more data selected from a group consisting of a Part number, Serial number, Manufacturer's lot number, Application name, Range, Date and time of each alarm, Start date and time of each service date, Date and time for each end of service time, Type of product, The cumulative exposure to the analyte, Manufacturing date, Shipping date, Unit of measure, Flow rate, Flow duration, Password or interface code and Expiration date.

9. The system of claim 2 wherein said end of service indicator includes one or more substances capable of interacting with the transformed substance and or trapped substances to cause a chemical change, a biochemical change, or a physical change to indicate the depletion, progress and end of service life of the transformer.

10. The system of claim 1 wherein said system for the detection and quantification of target analyte further comprises:
a transformer to trap xylene, toluene and other aliphatic hydrocarbons while allowing benzene to pass through and be detected by the sensing device;
a dryer to dry incoming fluids before said incoming fluids interacting with the transformer; and,
wherein the sensing device comprises a Photo ionization detector to detect the benzene passing though the transformer.

11. The system of claim 1 wherein said system for the detection and quantification of target analyte further comprises:
a transformer to trap xylene, toluene and other aliphatic hydrocarbons while allowing benzene to pass though and be detected by the sensing device;
a dryer to dry incoming fluids before interacting with the transformer; and,
a SAW detector to detect the benzene passing though the transformer.

12. The system of claim 1 further comprising:
a conditioning means arranged proximal to the transformer to assure that conversion of the target analyte to the more readily detectable second substance occurs at a particular temperature.

13. The system of claim 1 further comprising:
an RFID tag arranged on the transformer; and,
an RFID reader arranged proximal to the transformer for detecting information from the RFID tag.

14. The system of claim 1 further comprising:
a bar code that is arranged on the transformer, wherein the bar code includes information pertinent to the transformer;
a bar code reader arranged proximal to the bar code, said bar code reader decodes the information provided on the bar code;
a data processor that controls the pump and sensing unit, said data processor receiving data from the bar code reader; and,
a warning means that alerts an operator when a useful life of the transformer has expired.

15. A system for the detection and quantification of target analyte comprising;
a transformer having and fluid inlet and a fluid outlet and capable of transforming a target analyte that is hard to detect to a second substance where the second substance is capable of being detected by an existing sensing device, said first transformer comprising an end of service life indicator to indicate an end of service life of the first transformer, wherein the transformer localizes and traps undesired satellites of target analyte from passing through and being detected by a sensing device;
a sensing device connected to the fluid outlet of the transformer, said sensing device detects and quantifies the second substance to provide an accurate measure of the amount of target analyte taken from a sample; and,
a pump connected to one of the transformer or sensing device to draw the target analyte through the transformer and the second substance through the sensing device.

16. The system of claim 15 wherein said target analyte includes one or more substances selected from a group consisting of: Acetaldehyde, Acetone, Acetylene, Acrolein, Acrylonitrile, Arsine, Benzene, Bromine, Tert-Butyl Mercaptan, Carbon Disulfide, Carbon Monoxide, Carbon tetrachloride, Carbonyl Sulfide, Chlorine, Chlorobenzene, Chloroform, Diborane, o-Dichlorobenzene, 1,2-Dichloroethylene, Ethylene Glycol, Ethylene Oxide, Ethyl Mercaptan, Fluorine, Formaldehyde, Hydrocarbons (Aliphatic), Hydrogen Chloride, Hydrogen Bromide, Hydrogen Iodide, Hydrogen fluoride, Hydrogen Cyanide, Iodine, Mercaptans, Methacrylonitrile, Methyl Bromide, Nitrogen Oxides, Phosphine, Stoddard Solvent, Sulfur Dioxide, Tetrachloroethylene, Toluene, 1,1,1-Trichloroethane, Trichloroethylene, Vinyl Chloride, Vinylidene Chloride and Xylene.

17. The system of claim 15 wherein said sensing device is selected from a group consisting of Potentiometric sensors, Amperometric sensors, conductometric sensors, Acoustic IR sensors, Argon ion electron capture detectors, Biological sensors, Chemfets, Colorimetric sensors, Conductive polymer sensors, Enzyme sensors, Fiber optic sensors, Flame ionization detectors, Fluorescence detectors, immobilization of recombination bioluminescent bacterium sensors, Immunoassay sensors, Infrared coherent laser source sensors, Infrared sensors, Ion mobility detectors, Ionization sensors, Laser sensors, Metal oxide sensors, Paper tape sensors, Piezoelectric sensors, Pyroelectric sensors, Photo ionization sensors, Saw sensors, Solid state semi-conductor sensors, Solid state sensors, Solid state thin film semi-conductor sensors, TiO₂ sensors, Thermal conductivity sensors, Thin film titanium dioxide sensors, Voltametric sensors, electrocatalytic sensors and Wave guide sensors.

18. A system for the detection and quantification of target analyte comprising;
a transformer having and fluid inlet and a fluid outlet and capable of transforming a target analyte that is hard to detect to a second substance where the second substance is capable of being detected by an existing sensing device, said first transformer comprising an end of service life indicator to indicate an end of service life of the first transformer, wherein the transformer localizes and traps undesired satellites of target analyte from passing through and being detected by a sensing device;
a sensing device connected to the fluid outlet of the transformer, said sensing device detects and quantifies the second substance to provide an accurate measure of the amount of target analyte taken from a sample;
a pump connected to one of the transformer or sensing device to draw the target analyte through the transformer and the second substance through the sensing device; and,
a data processor that controls the pump and sensing unit, said data processor receiving data from the transformer that is used in controlling the pump and sensing unit.

19. The system of claim 18 wherein said target analyte includes one or more substances selected from a group consisting of: Acetaldehyde, Acetone, Acetylene, Acrolein, Acrylonitrile, Arsine, Benzene, Bromine, Tert-Butyl Mercaptan, Carbon Disulfide, Carbon Monoxide, Carbon tetrachloride, Carbonyl Sulfide, Chlorine, Chlorobenzene, Chloroform, Diborane, o-Dichlorobenzene, 1,2-Dichloroethylene, Ethylene Glycol, Ethylene Oxide, Ethyl Mercaptan, Fluorine, Formaldehyde, Hydrocarbons (Aliphatic), Hydrogen Chloride, Hydrogen Bromide, Hydrogen Iodide, Hydrogen fluoride, Hydrogen Cyanide, Iodine, Mercaptans, Methacrylonitrile, Methyl Bromide, Nitrogen Oxides, Phosphine, Stoddard Solvent, Sulfur Dioxide, Tetrachloroethylene, Toluene, 1,1,1-Trichloroethane, Trichloroethylene, Vinyl Chloride, Vinylidene Chloride and Xylene.

20. The system of claim 18 wherein said sensing device is selected from a group consisting of Potentiometric sensors, Amperometric sensors, conductometric sensors, Acoustic IR sensors, Argon ion electron capture detectors, Biological sensors, Chemfets, Colorimetric sensors, Conductive polymer sensors, Enzyme sensors, Fiber optic sensors, Flame ionization detectors, Fluorescence detectors, immobilization of recombination bioluminescent bacterium sensors, Immunoassay sensors, Infrared coherent laser source sensors, Infrared sensors, Ion mobility detectors, Ionization sensors, Laser sensors, Metal oxide sensors, Paper tape sensors, Piezoelectric sensors, Pyroelectric sensors, Photo ionization sensors, Saw sensors, Solid state semi-conductor sensors, Solid state sensors, Solid state thin film semi-conductor sensors, TiO₂ sensors, Thermal conductivity sensors, Thin film titanium dioxide sensors, Voltametric sensors, electrocatalytic sensors and Wave guide sensors.
